(19) United States
(12) Patent Application Publication
(10) Pub. No.: US 2025/0012884 A1
(43) Pub. Date: Jan. 9, 2025

(54) TECHNIQUES, SYSTEMS AND MACHINE READABLE PROGRAMS FOR MAGNETIC RESONANCE

(71) Applicant: Transmural Systems LLC, Andover, MA (US)

(72) Inventors: Ozgur Kocaturk, Andover, MA (US); Nasser Rafiee, Andover, MA (US)

(21) Appl. No.: 18/889,187

(22) Filed: Sep. 18, 2024

Related U.S. Application Data
(63) Continuation of application No. PCT/US2023/064715, filed on Mar. 20, 2023.
(60) Provisional application No. 63/321,579, filed on Mar. 18, 2022.
(60) Provisional application No. 63/373,537, filed on Aug. 25, 2022.

(51) Int. Cl. G01R 33/56; G01R 33/54; G01R 33/565

(52) U.S. Cl. CPC G01R 33/5608; G01R 33/546; G01R 33/565

(57) ABSTRACT

The present disclosure provides various methods and systems for performing magnetic resonance studies.

### CROSS-REFERENCE TO RELATED APPLICATIONS

The present patent application claims the benefit of priority to and is a continuation of International Patent Application No. PCT/US2023/064715, filed Mar. 20, 2023, which in turn claims the benefit of priority to U.S. Provisional Patent Application No. 63/321,579, filed Mar. 18, 2022, and U.S. Provisional Patent Application No. 63/373,537, filed Aug. 25, 2022. The present patent application is also related to U.S. patent application Ser. No. 17/238,897 field Apr. 23, 2021. Each of the foregoing patent applications is incorporated by reference herein in its entirety for all purposes.

### BACKGROUND OF THE DISCLOSURE

Interventional magnetic resonance imaging (MRI) can, at least in theory, allow for accurate diagnosis and treatment, due to its superior soft tissue contrast and ionizing radiation free imaging modality. However, the magnetic and electric fields in the MRI scanner makes using the standard guidewires and catheters almost impossible. The present disclosure provides solutions to these problems, as set forth below.

### SUMMARY OF THE DISCLOSURE

Advantages of the present disclosure will be set forth in and become apparent from the description that follows. Additional advantages of the disclosure will be realized and attained by the methods and systems particularly pointed out in the written description and claims hereof, as well as from the appended drawings.

In accordance with the disclosure, methods (and systems for performing the methods and non-transitory computer readable mediums for performing the methods) are provided as set forth below.

In accordance with some aspects, a method for performing magnetic resonance imaging. The method includes providing a magnetic resonance device including (i) a main magnet for providing a background magnetic field along a first direction, (ii) at least one radio-frequency coil, and (iii) at least one gradient coil that can be controlled to define a region of interest. The method further includes positioning a medical device with at least one MRI passive or active marker thereon within a field of view of the at least one resonant coil, the medical device being configured to emit a signal either passively or actively. The method still further includes providing a circuit that generates pulsed DC current or AC current to act as a signal signature for the MRI passive or active marker on the medical device, the circuit being further configured to adjust signal parameters including at least one of the signal amplitude, pulse shape and signal repetition rate relative to MR scan sequence parameters to permit the at least one MRI passive or active marker to emit a signal to facilitate detection of the medical device. The method still further includes introducing a sample or subject to be studied into the field of view, introducing RF pulses into the sample or subject, collecting data for a set of nuclei of interest from the sample or subject, and the passive or active marker. The method still further includes processing the data to determine the spatial location of the medical device based on the passive or active marker, and forming image data showing a relative location of the medical device with respect to surrounding anatomy.

If desired, the same medical device can be configured to be used in different magnetic field strengths and different MR sequences without sacrificing medical image quality and precision for detecting device position.

The disclosure further provides machine readable programs, systems and methods of performing magnetic resonance imaging that includes delivering pulsed DC current or AC current waveforms to create a dedicated signature in the form of magnetic field inhomogeneity at a location of a conductive markers on a medical device located within a magnetic resonance imaging device.

For example, if desired, the pulsed DC or AC current can be delivered in a manner that accounts for the TR/TE values of a MRI imaging sequence. If desired, an algorithm can be applied to a signal trail received at a magnetic resonance device to identify the magnetic field inhomogeneities. If desired, the magnetic field inhomogeneities can be dynamically adjustable magnetic field disruptions. The dynamically adjustable magnetic field disruptions can be interspersed in predetermined intervals within MR signals received by the magnetic resonance device.

In further accordance with the disclosure, if desired, when the signals that are generated by the conductive markers are detected by the algorithm, the spatial location of these magnetic field inhomogeneities can then be determined in real time. If desired, image reconstruction software of the magnetic resonance device can be configured with instructions to colorize and/or superimpose an image of the medical device on the anatomical image after transforming received MRI signals to medical images during real time MRI. Moreover, different marker signatures can be created and switched during real time MRI. In some implementations, it is possible for a physician to turn off the passive marker signal to view the anatomical image without any magnetic field disruption.

In further accordance with the disclosure, a low pulsed DC current can be applied to the medical device or marker in the range of 1-1000 Hz. If desired, the low pulsed DC current is between 50-200 mA. Moreover, a low pulsed AC current can additionally or alternatively be applied to the medical device or marker in the range of 1-1000 Hz. If desired, the low pulsed AC current can be between 50-200 mA.

In further accordance with the disclosure, current applied to the medical device or marker can be applied using a frequency outside of the imaging bandwidth but within the MRI receiver coil bandwidth to create a predetermined signal signature for the magnetic field inhomogeneities. If desired, the pulsed DC or AC current can be between about 0.5V and about 12.0V. In accordance with some aspects, software used to detect the presence of medical devices or markers can utilize a machine learning (“ML”) algorithm to look for dynamically adjustable magnetic field disruptions in the imaging plane or specific frequency shift in k-space domain.

As set forth herein, systems are provided to perform any of the disclosed methods. Moreover, non-transitory computer readable media storing a computer program to operate a MRI system are disclosed to perform any of the disclosed methods

It is to be understood that the following description is exemplary and is intended to provide further explanation of the disclosed embodiments. The accompanying drawings, which are incorporated in and constitute part of this specification, are included to illustrate and provide a further understanding of the disclosed methods and systems. Together with the description, the drawings serve to explain principles of the disclosure.

### BRIEF DESCRIPTION OF THE DRAWINGS

FIG. 1 illustrates a first illustrative circuit in accordance with the disclosure.

FIG. 2 illustrates a second illustrative circuit in accordance with the disclosure.

FIG. 3 illustrates an example of a MR scanner system.

FIG. 4 illustrates aspects of a computer coordinator in accordance with the present disclosure.

### DETAILED DESCRIPTION OF PREFERRED EMBODIMENTS

Reference will now be made in detail to the present preferred embodiments of the disclosure, examples of which are illustrated in the accompanying drawings. The methods and corresponding steps of the disclosed embodiments will be described in conjunction with the detailed description of the system.

Interventional magnetic resonance imaging (MRI) can potentially allow for accurate diagnosis and treatment, due to its superior soft tissue contrast and ionizing radiation free imaging modality. However, the magnetic and electric fields in the MRI scanner makes using standard guidewires and catheters almost impossible.

Dedicated MRI conditional or safe medical devices are needed to perform interventional procedures under MRI safely. Tracking or profiling markers are needed to make the tip and shaft of the medical devices visible Also, these markers should have a low profile and flexible enough to navigate safely in small tortuous vessels.

There are three main methods for MRI markers have been broadly categorized in the interventional MRI literature as passive, active and semi-active. Passive markers such as iron oxide or paramagnetic stainless steel cause signal void or their intrinsic magnetic susceptibility cause local B₀ field inhomogeneities to track the interventional devices such as guidewires and needles [1].

Active markers are usually solenoid coils or monopole antennas embedded into the medical device shaft including transmission line along the device shaft to transmit the received RF signal including the active markers' spatial information to the scanner. Active visualization techniques require modifying existing scanner coil files to introduce the active markers as an internal MR scanner coil so that the received RF signal can be combined with RF signal received through external coils to construct anatomical images with medical devices. Also, additional radio frequency cables might interfere with the maneuverability of the medical devices depending on the clinical application.

Semi active markers are basically LC resonant markers. Several different variations are reported in the literature [2-8]. The semi active markers inductively couple with the magnetic flux from the radiofrequency (RF) transmission pulse, resulting in induced current in the markers, and this induced current create a local magnetic field [9]. The locally amplified B₁+ field produces higher flip angles in close vicinity with these markers, then enhances signal or alters contrast in anatomical images taken under MRI.

Although the semi active device visualization technique is simpler than the active visualization technique, its performance strongly depends on the device size and orientation relative to the main magnetic field. In deeper tissue applications such as cardiovascular procedures, the inductive coupling between the semi active markers and the external MR coils weakens and alters contrast adversely [10].

The third category is the passive markers that are incorporated into the device construction to enhance, reduce or distort the T1 and or T2 weighted MRI signal in close vicinity of the medical device within the body. Ferromagnetic marker materials such as stainless steel or ferrimagnetic marker materials such as copper zinc ferrite can be used to enhance the contrast between the markers and background tissues for device visualization purpose [11,12]. However, in this technique the device visualization depends on the contrast to noise ratio (CNR) which also depends on the susceptibility artifact induced by the marker and also imaging sequence parameters. Especially for ferromagnetic materials there is a nonlinear relationship between the artifact size and magnetic field strength [13]. Therefore, it is almost impossible to have constant size artifact from the markers through real time MRI.

As an alternative to the positive contrast passive markers is the application of small direct currents (DCs)(10-150 mA) running through RF loop coils embedded into the device body [14,15]. In this method, DC current application creates magnetic fields that disturb the magnetic field homogeneity around the markers (i.e. loop coils) and provides device tip visibility. However, continuous DC current may cause ohmic heating along the device [16].

In accordance with some implementations, variations of novel medical device tracking methods and related devices and systems are provided that can eliminate many, if not all, of the disadvantages of the existing techniques.

RF antenna patterns and transmission lines formed over the medical device shaft (e.g, printed via conductive ink or deposited by way of physical vapor deposition) can be energized to create local magnetic field homogeneities that can cause local contrast (and thereby function as a passive marker) to visualize the medical device during real time MRI. Existing techniques create magnetic field inhomogeneities using DC current only. However, when only DC current is applied it can create continuous magnetic field disruption, and the susceptibility artifact size can vary based on the medical device orientation relative to the main magnetic field of the MR scanner and MRI scan parameters. Therefore, it can be quite challenging to distinguish passive markers from the anatomy during real time MRI.

In further accordance with the disclosure, it is possible to provide low pulsed DC current (e.g., 1-1000 Hz (or any increment of 1 Hz therebetween or subrange of 50 Hz therebetween), 50-200 mA DC current (or any increment of 1 mA therebetween or any subrange of 10 mA therebetween)) or AC current (e.g, 50-200 mA) (or any increment of 1 mA therebetween or any subrange of 10 mA therebetween) with frequencies outside of the imaging bandwidth but within the MRI receiver coil bandwidth to create a predetermined signal signature for the magnetic field inhomogeneities around loop coils that can be provided along the medical device shaft. The pulsed frequency values can be established in accordance with the TR/TE values of the MRI sequence used for the imaging or the Larmour frequency of the MRI scanner, wherein Repetition Time (TR) is the amount of time between successive pulse sequences applied to the same slice, and time to Echo (TE) is the time between the delivery of the RF pulse and the receipt of the echo signal. The voltage of the applied waveform can vary, for example, between about 0.5V and about 12.0V, any increment therein of about 0.5V, or any subrange therein of 1 to 5 Volts.

A machine learning (“ML”) algorithm developed and embedded into image processing software can be configured to look for these dynamically adjustable magnetic field disruptions in the imaging plane or specific frequency shift in k-space domain. This can be expected to occur in predetermined intervals within the received MR signals. When the signals that are then generated by the medical devices or the susceptibility artifact are detected by the ML algorithm in frequency domain or image domain respectively, the spatial location of these magnetic field inhomogeneities can be determined (e.g., their x,y,z coordinates) in real time.

Then, if desired, the image reconstruction software can be configured with instructions to colorize and superimpose an image of the invasive instrument onto the anatomical image after transforming received MRI signal to medical images during real time MRI. The advantage of this illustrative technique is that it provides colorized passive marker imaging without any cable connection to the MRI scanner. Moreover, it is also very flexible to apply MR scanners with different magnetic field strengths. For different MRI sequences, different passive marker signatures can be created and switched during real time MRI. Also, the physician can selectively turn off or turn on the passive marker signal anytime when he/she wants to focus on the anatomical image without any magnetic field disruption.

The pulse generator that creates pulsed DC current or AC current with frequency(ies) close to the Larmour frequency (outside of the imaging bandwidth but within the MRI receiver coil bandwidth) can be powered by a MRI compatible battery or by the main power of the MRI scanner. The current type (pulsed DC or AC) and the frequency can be selected based on the MRI scan sequence and scan parameters. The pulse generator can receive TTL signal from the scanner to synchronize the applied current to the RF transmission phase of the MRI scanner when needed. Transistor-transistor logic (TTL) is a digital logic design in which bipolar transistor s act on direct-current pulses. After the MRI scanner receives the analog RF signal, the computing system and digital processor(s) of the MRI scanner performs analog/digital conversion, and the digitized imaging data is shared with a computer running a customized ML algorithm. When the image construction computer of the scanner processes the acquired data to form medical images, the ML algorithm processes the same data for detecting the spatial location of the device markers only (in frequency domain or imaging domain). After detecting the susceptibility artifact locations, the ML algorithm can colorize these markers and superimpose them onto the medical images then the final images including both anatomical imaging and passive marker locations can be seen on MR image display.

In accordance with further implementations, different RF antenna geometries (preferably resonant antenna geometries) can be designed based on the device profile and targeted clinical application and then printed on the medical device shaft, for example, as a single or multiple layers using conductive ink such as silver based inks or carbon inks. Both inductance-related and capacitance-related features of the RF resonant antennas can be distributed between layers. If desired, conductive layers can be separated using thin wall polymer tubings or dielectric inks. Transmission line(s) can also be printed using conductive ink to transmit the received RF signal to the amplification circuit as set forth in the patent application annexed hereto.

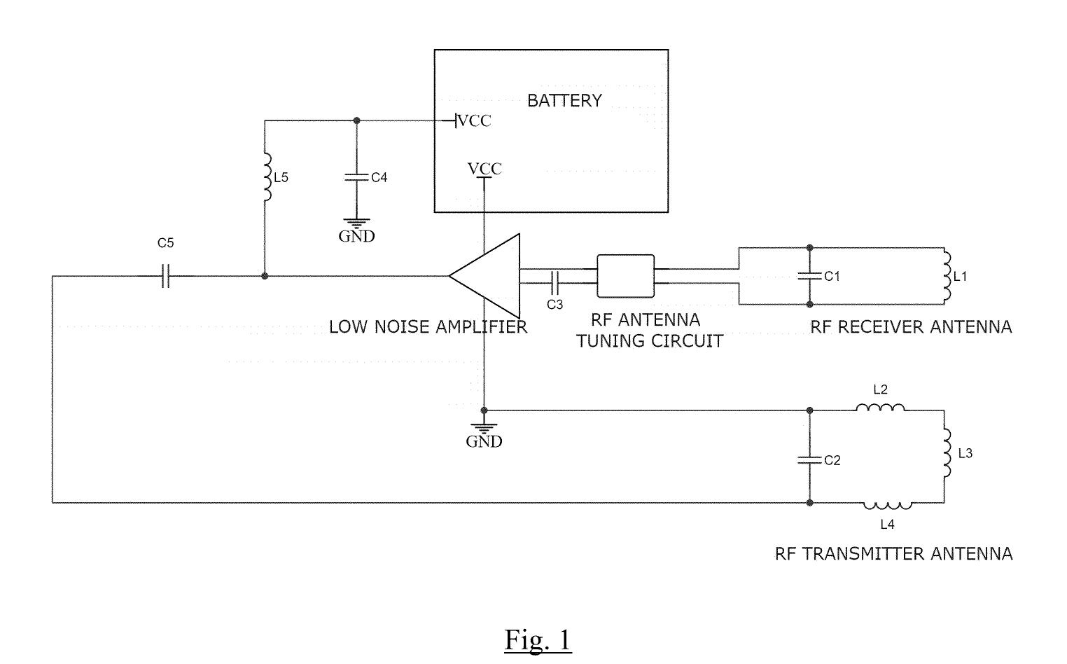

An illustrative amplification circuit depicted in FIG. 1 can include three subunits. A first sub unit, or circuit, can include tuning circuitry to match the overall RF receiver antenna to the Larmour frequency of the MR scanner to enhance the antenna performance such as by increasing the signal-to-noise ratio (“SNR”). More specifically, FIG. 1 depicts an illustrative double layer wireless semi active RF antenna design. The received RF signal can be amplified and then fed into an additional antenna that can couple with external RF coils of the MRI scanner.

A second sub-unit, or circuit can include a low power low noise high gain amplifier to amplify the received RF signal and feed back to the RF receiver antenna.

A third subunit, or circuit, can include a MRI compatible battery and voltage regulation circuitry to power the amplifier. The frequency content of the received RF signal can contain the spatial information of the RF antenna. The amplified signal can be fed to the same RF receiver antenna or to a separate inductive coupling coil printed on outermost separate layer. The amplifier can be powered by a MRI compatible battery including voltage and/or current regulation circuitry to ensure reliable performance and overall safety. A fiberoptic transmitter and receiver system (that converts the radio frequency signal to optical signal) can be added to the system to eliminate the radio frequency interference during transmitting the received MR signal to the low noise amplifier and also to transmit the amplified signal back to the RF transmitter antenna.

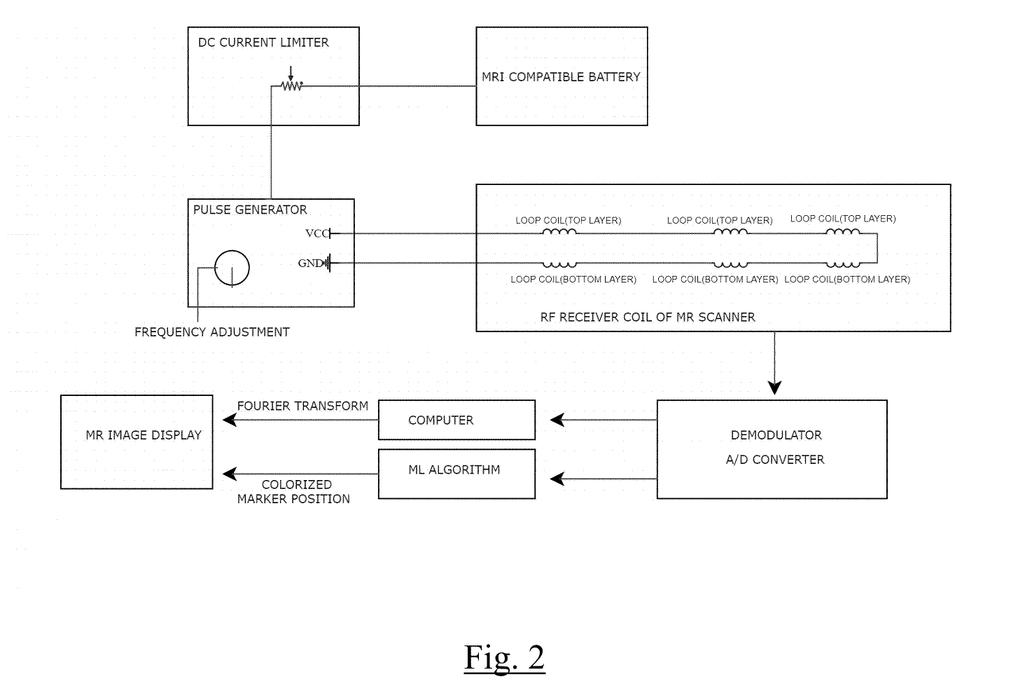

FIG. 2 depicts a single or multiple layer RF antenna structure can be printed on the medical device shaft and be energized via pulsed DC current or AC current to create specific magnetic field inhomogeneities to act as a position detection signature for the medical device.

### Exemplary MRI Scanner Systemization

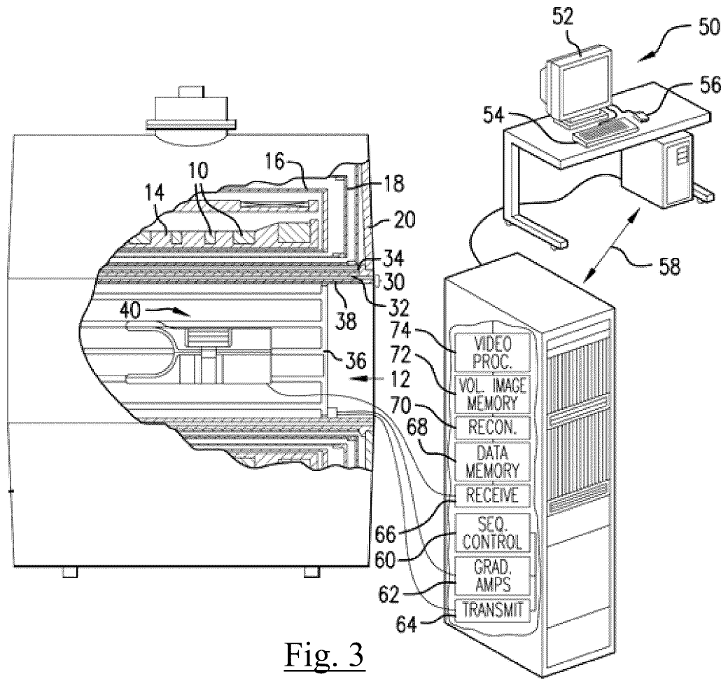

An exemplary magnetic resonance system is depicted in FIG. 3, and includes a plurality of primary magnetic coils 10 that generate a uniform, temporally constant magnetic field B₀ along a longitudinal or z-axis of a central bore 12 of the device. In a preferred superconducting embodiment, the primary magnet coils are supported by a former 14 and received in a toroidal helium vessel or can 16. The vessel is filled with helium to maintain the primary magnet coils at superconducting temperatures. For low tesla scanners greatly reduce the amount of helium needed for cooling the magnet system. The can is surrounded by a series of cold shields 18 which are supported in a vacuum Dewar 20. Of course, annular resistive magnets, C-magnets, and the like are also contemplated.

A whole body gradient coil assembly 30 includes x, y, and z-coils mounted along the bore 12 for generating gradient magnetic fields, Gx, Gy, and Gz. Preferably, the gradient coil assembly is a self-shielded gradient coil that includes primary x, y, and z-coil assemblies 32 potted in a dielectric former and secondary x, y, and z-coil assemblies 34 that are supported on a bore defining cylinder of the vacuum Dewar 20. A whole body radio frequency coil 36 can be mounted inside the gradient coil assembly 30. A whole body radio frequency shield 38, e.g., copper mesh, can be mounted between the whole body RF coil 36 and the gradient coil assembly 30. If desired, an insertable radio frequency coil 40 can be removably mounted in the bore in an examination region defined around an isocenter of the magnet 10. In the embodiment of FIG. 2, the insertable radio frequency coil is a head and neck coil for imaging one or both of patient's head and neck, but other extremity coils can be provided, such as back coils for imaging the spine, knee coils, shoulder coils, breast coils, wrist coils and the like.

With continuing reference to FIG. 3, an operator interface and control station is provided that includes a human-readable display, such as a video monitor 52, and operator input devices such as a keyboard 54, a mouse 56, a trackball, light pen, or the like. A computer control and reconstruction module 58 is also provided that includes hardware and software for enabling the operator to select among a plurality of preprogrammed magnetic resonance sequences that are stored in a sequence control memory, if rf pulses are to be used as a part of the imaging study. A sequence controller 60 controls gradient amplifiers 62 connected with the gradient coil assembly 30 for causing the generation of the Gx, Gy, and Gz gradient magnetic fields at appropriate times during the selected gradient sequence and a digital transmitter 64 which causes a selected one of the whole body and insertable radio frequency coils to generate B₁ radio frequency field pulses at times appropriate to the selected sequence, if rf pulses are to be used in the study.

MR signals received by the coil 40 are demodulated by a digital receiver 66 and stored in a data memory 68. The data from the data memory are reconstructed by a reconstruction or array processor 70 into a volumetric image representation that is stored in an image memory 72. If a phased array is used as the receiving coil assembly, the image can be reconstructed from the coil signals. A video processor 74 under operator control converts selected portions of the volumetric image representation into slice images, projection images, perspective views, or the like as is conventional in the art for display on the video monitor.

The disclosed embodiments of MRI visible devices can be incorporated into such a MR scanner by providing additional software to detect the signals from the medical devices which are physicially connected to the MR scanner patient table via RF coax cables and superimpose them on the image in real time as set forth above.

The disclosed medical devices and circuitry can be configured to be used in different magnetic field strengths (e.g., between 0.1 Tesla and 10.0 Tesla background field in increments of 0.1 Tesla) and different MR sequences without sacrificing medical image quality and precision for detecting device position.

### Example—TSMS™ Controller

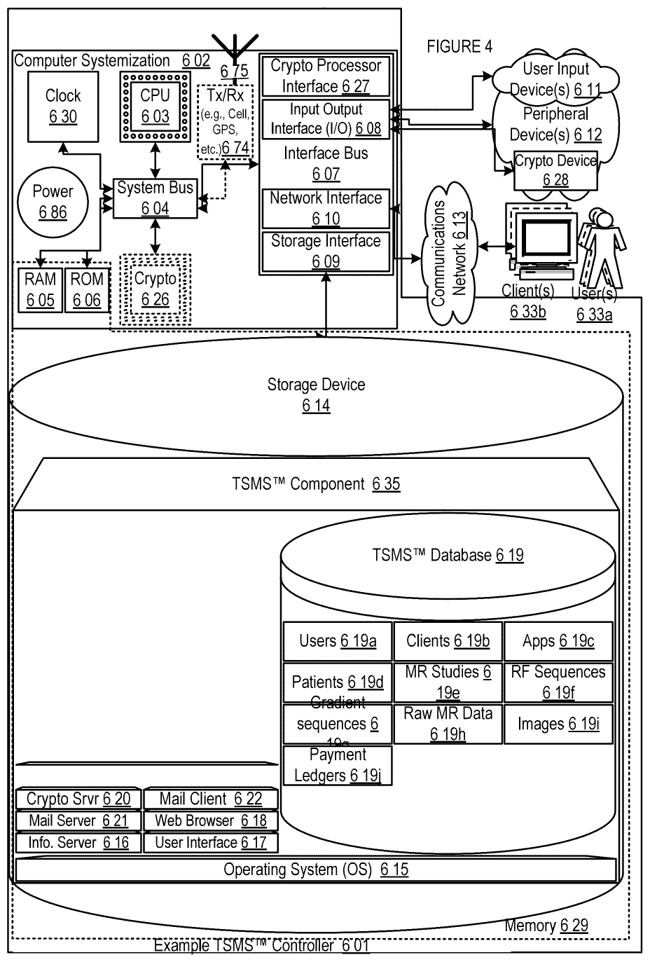

FIG. 4 illustrates inventive aspects of a TSMS™ controller 601 for controlling a system such as that illustrated in FIG. 6 implementing some of the embodiments disclosed herein. In this embodiment, the TSMS™ controller 601 may serve to aggregate, process, store, search, serve, identify, instruct, generate, match, and/or facilitate interactions with a computer through various technologies, and/or other related data.

With respect to the controller 601, typically, a user or users, e.g., 633a, which may be people or groups of users and/or other systems, may engage information technology systems (e.g., computers) to facilitate operation of the system and information processing. In turn, computers employ processors to process information; such processors 603 may be referred to as central processing units (CPU). One form of processor is referred to as a microprocessor. CPUs use communicative circuits to pass binary encoded signals acting as instructions to enable various operations. These instructions may be operational and/or data instructions containing and/or referencing other instructions and data in various processor accessible and operable areas of memory 629 (e.g., registers, cache memory, random access memory, etc.). Such communicative instructions may be stored and/or transmitted in batches (e.g., batches of instructions) as programs and/or data components to facilitate desired operations. These stored instruction codes, e.g., programs, may engage the CPU circuit components and other motherboard and/or system components to perform desired operations. One type of program is a computer operating system, which, may be executed by CPU on a computer; the operating system enables and facilitates users to access and operate computer information technology and resources. Some resources that may be employed in information technology systems include: input and output mechanisms through which data may pass into and out of a computer; memory storage into which data may be saved; and processors by which information may be processed. These information technology systems may be used to collect data for later retrieval, analysis, and manipulation, which may be facilitated through a database program. These information technology systems provide interfaces that allow users to access and operate various system components.

In one embodiment, the TSMS™ controller 601 may be connected to and/or communicate with entities such as, but not limited to: one or more users from user input devices 611; peripheral devices 612, components of the magnetic resonance system; an optional cryptographic processor device 628; and/or a communications network 613. For example, the TSMS™ controller 601 may be connected to and/or communicate with users, e.g., 633a, operating client device(s), e.g., 633b, including, but not limited to, personal computer(s), server(s) and/or various mobile device(s) including, but not limited to, cellular telephone(s), smartphone(s) (e.g., iPhone®, Blackberry®, Android OS-based phones etc.), tablet computer(s) (e.g., Apple iPad™, HP Slate™, Motorola Xoom™, etc.), eBook reader(s) (e.g., Amazon Kindle™, Barnes and Noble's Nook™ eReader, etc.), laptop computer(s), notebook(s), netbook(s), gaming console(s) (e.g., XBOX Live™, Nintendo® DS, Sony PlayStation® Portable, etc.), portable scanner(s) and/or the like.

Networks are commonly thought to comprise the interconnection and interoperation of clients, servers, and intermediary nodes in a graph topology. It should be noted that the term “server” as used throughout this application refers generally to a computer, other device, program, or combination thereof that processes and responds to the requests of remote users across a communications network. Servers serve their information to requesting “clients.” The term “client” as used herein refers generally to a computer, program, other device, user and/or combination thereof that is capable of processing and making requests and obtaining and processing any responses from servers across a communications network. A computer, other device, program, or combination thereof that facilitates, processes information and requests, and/or furthers the passage of information from a source user to a destination user is commonly referred to as a “node.” Networks are generally thought to facilitate the transfer of information from source points to destinations. A node specifically tasked with furthering the passage of information from a source to a destination is commonly called a “router.” There are many forms of networks such as Local Area Networks (LANs), Pico networks, Wide Area Networks (WANs), Wireless Networks (WLANs), etc. For example, the Internet is generally accepted as being an interconnection of a multitude of networks whereby remote clients and servers may access and interoperate with one another.

The TSMS™ controller 601 may be based on computer systems that may comprise, but are not limited to, components such as: a computer systemization 602 connected to memory 629.

### Computer Systemization

A computer systemization 602 may comprise a clock 630, central processing unit (“CPU(s)” and/or “processor(s)” (these terms are used interchangeable throughout the disclosure unless noted to the contrary)) 603, a memory 629 (e.g., a read only memory (ROM) 606, a random access memory (RAM) 605, etc.), and/or an interface bus 607, and most frequently, although not necessarily, are all interconnected and/or communicating through a system bus 604 on one or more (mother)board(s) 602 having conductive and/or otherwise transportive circuit pathways through which instructions (e.g., binary encoded signals) may travel to effect communications, operations, storage, etc. Optionally, the computer systemization may be connected to an internal power source 686; e.g., optionally the power source may be internal. Optionally, a cryptographic processor 626 and/or transceivers (e.g., ICs) 674 may be connected to the system bus. In another embodiment, the cryptographic processor and/or transceivers may be connected as either internal and/or external peripheral devices 612 via the interface bus I/O. In turn, the transceivers may be connected to antenna(s) 675, thereby effectuating wireless transmission and reception of various communication and/or sensor protocols; for example the antenna(s) may connect to: a Texas Instruments WiLink WL1283 transceiver chip (e.g., providing 802.11n, Bluetooth 3.0, FM, global positioning system (GPS) (thereby allowing TSMS™ controller to determine its location)); Broadcom BCM4329FKUBG transceiver chip (e.g., providing 802.11n, Bluetooth 2.1+EDR, FM, etc.); a Broadcom BCM4750IUB8 receiver chip (e.g., GPS); an Infineon Technologies X-Gold 618-PMB9800 (e.g., providing 2G/3G HSDPA/HSUPA communications); and/or the like. The system clock typically has a crystal oscillator and generates a base signal through the computer systemization's circuit pathways. The clock is typically coupled to the system bus and various clock multipliers that will increase or decrease the base operating frequency for other components interconnected in the computer systemization. The clock and various components in a computer systemization drive signals embodying information throughout the system. Such transmission and reception of instructions embodying information throughout a computer systemization may be commonly referred to as communications. These communicative instructions may further be transmitted, received, and the cause of return and/or reply communications beyond the instant computer systemization to: communications networks, input devices, other computer systemizations, peripheral devices, and/or the like. Of course, any of the above components may be connected directly to one another, connected to the CPU, and/or organized in numerous variations employed as exemplified by various computer systems.

The CPU comprises at least one high-speed data processor adequate to execute program components for executing user and/or system-generated requests. Often, the processors themselves will incorporate various specialized processing units, such as, but not limited to: integrated system (bus) controllers, memory management control units, floating point units, and even specialized processing sub-units like graphics processing units, digital signal processing units, and/or the like. Additionally, processors may include internal fast access addressable memory, and be capable of mapping and addressing memory 629 beyond the processor itself; internal memory may include, but is not limited to: fast registers, various levels of cache memory (e.g., level 1, 2, 3, etc.), RAM, etc. The processor may access this memory through the use of a memory address space that is accessible via instruction address, which the processor can construct and decode allowing it to access a circuit path to a specific memory address space having a memory state. The CPU may be a microprocessor such as: AMD's Athlon, Duron and/or Opteron; ARM's application, embedded and secure processors; IBM and/or Motorola's DragonBall and PowerPC; IBM's and Sony's Cell processor; Intel's Celeron, Core (2) Duo, Itanium, Pentium, Xeon, and/or XScale; and/or the like processor(s). The CPU interacts with memory through instruction passing through conductive and/or transportive conduits (e.g., (printed) electronic and/or optic circuits) to execute stored instructions (i.e., program code) according to conventional data processing techniques. Such instruction passing facilitates communication within the TSMS™ controller and beyond through various interfaces. Should processing requirements dictate a greater amount speed and/or capacity, distributed processors (e.g., Distributed TSMS™ embodiments), mainframe, multi-core, parallel, and/or super-computer architectures may similarly be employed. Alternatively, should deployment requirements dictate greater portability, smaller Personal Digital Assistants (PDAs) may be employed.

Depending on the particular implementation, features of the TSMS™ implementations may be achieved by implementing a microcontroller such as CAST's R8051XC2 microcontroller; Intel's MCS 51 (i.e., 8051 microcontroller); and/or the like. Also, to implement certain features of the TSMS™ embodiments, some feature implementations may rely on embedded components, such as: Application-Specific Integrated Circuit (“ASIC”), Digital Signal Processing (“DSP”), Field Programmable Gate Array (“FPGA”), and/or the like embedded technology. For example, any of the TSMS™ component collection (distributed or otherwise) and/or features may be implemented via the microprocessor and/or via embedded components; e.g., via ASIC, coprocessor, DSP, FPGA, and/or the like. Alternately, some implementations of the TSMS™ may be implemented with embedded components that are configured and used to achieve a variety of features or signal processing.

Depending on the particular implementation, the embedded components may include software solutions, hardware solutions, and/or some combination of both hardware/software solutions. For example, TSMS™ features discussed herein may be achieved through implementing FPGAs, which are a semiconductor devices containing programmable logic components called “logic blocks”, and programmable interconnects, such as the high performance FPGA Virtex series and/or the low cost Spartan series manufactured by Xilinx. Logic blocks and interconnects can be programmed by the customer or designer, after the FPGA is manufactured, to implement any of the TSMS™ features. A hierarchy of programmable interconnects allow logic blocks to be interconnected as needed by the TSMS™ system designer/administrator, somewhat like a one-chip programmable breadboard. An FPGA's logic blocks can be programmed to perform the function of basic logic gates such as AND, and XOR, or more complex combinational functions such as decoders or simple mathematical functions. In most FPGAs, the logic blocks also include memory elements, which may be simple flip-flops or more complete blocks of memory. In some circumstances, the TSMS™ may be developed on regular FPGAs and then migrated into a fixed version that more resembles ASIC implementations. Alternate or coordinating implementations may migrate TSMS™ controller features to a final ASIC instead of or in addition to FPGAs. Depending on the implementation all of the aforementioned embedded components and microprocessors may be considered the “CPU” and/or “processor” for the TSMS™

### Power Source

The power source 686 may be of any standard form for powering small electronic circuit board devices such as the following power cells: alkaline, lithium hydride, lithium ion, lithium polymer, nickel cadmium, solar cells, and/or the like. Other types of AC or DC power sources may be used as well. In the case of solar cells, in one embodiment, the case provides an aperture through which the solar cell may capture photonic energy. The power cell 686 is connected to at least one of the interconnected subsequent components of the TSMS™ thereby providing an electric current to all subsequent components. In one example, the power source 686 is connected to the system bus component 604. In an alternative embodiment, an outside power source 686 is provided through a connection across the I/O 608 interface. For example, a USB and/or IEEE 1394 connection carries both data and power across the connection and is therefore a suitable source of power.

### Interface Adapters

Interface bus(ses) 607 may accept, connect, and/or communicate to a number of interface adapters, conventionally although not necessarily in the form of adapter cards, such as but not limited to: input output interfaces (I/O) 608, storage interfaces 609, network interfaces 610, and/or the like. Optionally, cryptographic processor interfaces 627 similarly may be connected to the interface bus. The interface bus provides for the communications of interface adapters with one another as well as with other components of the computer systemization. Interface adapters are adapted for a compatible interface bus. Interface adapters conventionally connect to the interface bus via a slot architecture. Conventional slot architectures may be employed, such as, but not limited to: Accelerated Graphics Port (AGP), Card Bus, (Extended) Industry Standard Architecture ((E)ISA), Micro Channel Architecture (MCA), NuBus, Peripheral Component Interconnect (Extended) (PCI(X)), PCI Express, Personal Computer Memory Card International Association (PCMCIA), and/or the like.

Storage interfaces 609 may accept, communicate, and/or connect to a number of storage devices such as, but not limited to: storage devices 614, removable disc devices, and/or the like. Storage interfaces may employ connection protocols such as, but not limited to: (Ultra) (Serial) Advanced Technology Attachment (Packet Interface) ((Ultra) (Serial) ATA(PI)), (Enhanced) Integrated Drive Electronics ((E)IDE), Institute of Electrical and Electronics Engineers (IEEE) 1394, fiber channel, Small Computer Systems Interface (SCSI), Universal Serial Bus (USB), and/or the like.

Network interfaces 610 may accept, communicate, and/or connect to a communications network 613. Through a communications network 613, the TSMS™ controller is accessible through remote clients 633b (e.g., computers with web browsers) by users 633a. Network interfaces may employ connection protocols such as, but not limited to: direct connect, Ethernet (thick, thin, twisted pair 10/100/1000 Base T, and/or the like), Token Ring, wireless connection such as IEEE 802.11a-x, and/or the like. Should processing requirements dictate a greater amount speed and/or capacity, distributed network controllers (e.g., Distributed TSMS™), architectures may similarly be employed to pool, load balance, and/or otherwise increase the communicative bandwidth required by the TSMS™ controller. A communications network may be any one and/or the combination of the following: a direct interconnection; the Internet; a Local Area Network (LAN); a Metropolitan Area Network (MAN); an Operating Missions as Nodes on the Internet (OMNI); a secured custom connection; a Wide Area Network (WAN); a wireless network (e.g., employing protocols such as, but not limited to a Wireless Application Protocol (WAP), I-mode, and/or the like); and/or the like. A network interface may be regarded as a specialized form of an input output interface. Further, multiple network interfaces 610 may be used to engage with various communications network types 613. For example, multiple network interfaces may be employed to allow for the communication over broadcast, multicast, and/or unicast networks.

Input Output interfaces (I/O) 608 may accept, communicate, and/or connect to user input devices 611, peripheral devices 612, cryptographic processor devices 628, and/or the like. I/O may employ connection protocols such as, but not limited to: audio: analog, digital, monaural, RCA, stereo, and/or the like; data: Apple Desktop Bus (ADB), IEEE 1394a-b, serial, universal serial bus (USB); infrared; joystick; keyboard; midi; optical; PC AT; PS/2; parallel; radio; video interface: Apple Desktop Connector (ADC), BNC, coaxial, component, composite, digital, Digital Visual Interface (DVI), high-definition multimedia interface (HDMI), RCA, RF antennae, S-Video, VGA, and/or the like; wireless transceivers: 802.11a/b/g/n/x; Bluetooth; cellular (e.g., code division multiple access (CDMA), high speed packet access (HSPA(+)), high-speed downlink packet access (HSDPA), global system for mobile communications (GSM), long term evolution (LTE), WiMax, etc.); and/or the like. One typical output device may include a video display, which typically comprises a Cathode Ray Tube (CRT) or Liquid Crystal Display (LCD) based monitor with an interface (e.g., DVI circuitry and cable) that accepts signals from a video interface, may be used. The video interface composites information generated by a computer systemization and generates video signals based on the composited information in a video memory frame. Another output device is a television set, which accepts signals from a video interface. Typically, the video interface provides the composited video information through a video connection interface that accepts a video display interface (e.g., an RCA composite video connector accepting an RCA composite video cable; a DVI connector accepting a DVI display cable, etc.).

User input devices 611 often are a type of peripheral device 612 (see below) and may include: card readers, dongles, fingerprint readers, gloves, graphics tablets, joysticks, keyboards, microphones, mouse (mice), remote controls, retina readers, touch screens (e.g., capacitive, resistive, etc.), trackballs, trackpads, sensors (e.g., accelerometers, ambient light, GPS, gyroscopes, proximity, etc.), styluses, and/or the like.

Peripheral devices 612, such as other components of the MR system, including signal generators in communication with RF coils, receivers in communication with RF coils, the gradient coil system, main magnet system and the like may be connected and/or communicate to I/O and/or other facilities of the like such as network interfaces, storage interfaces, directly to the interface bus, system bus, the CPU, and/or the like. Peripheral devices may be external, internal and/or part of the TSMS™ controller. Peripheral devices may also include: antenna, audio devices (e.g., line-in, line-out, microphone input, speakers, etc.), cameras (e.g., still, video, webcam, etc.), dongles (e.g., for copy protection, ensuring secure transactions with a digital signature, and/or the like), external processors (for added capabilities; e.g., crypto devices 628), force-feedback devices (e.g., vibrating motors), network interfaces, printers, scanners, storage devices, transceivers (e.g., cellular, GPS, etc.), video devices (e.g., goggles for functional imaging, for example, monitors, etc.), video sources, visors, and/or the like. Peripheral devices often include types of input devices (e.g., cameras).

Cryptographic units such as, but not limited to, microcontrollers, processors 626, interfaces 627, and/or devices 628 may be attached, and/or communicate with the TSMS™ controller. A MC68HC16 microcontroller, manufactured by Motorola Inc., may be used for and/or within cryptographic units. The MC68HC16 microcontroller utilizes a 16-bit multiply-and-accumulate instruction in the 16 MHz configuration and requires less than one second to perform a 512-bit RSA private key operation. Cryptographic units support the authentication of communications from interacting agents, as well as allowing for anonymous transactions. Cryptographic units may also be configured as part of CPU. Equivalent microcontrollers and/or processors may also be used. Other commercially available specialized cryptographic processors include: the Broadcom's CryptoNetX and other Security Processors; nCipher's nShield, SafeNet's Luna PCI (e.g., 7100) series; Semaphore Communications' 40 MHz Roadrunner 184; Sun's Cryptographic Accelerators (e.g., Accelerator 6000 PCIe Board, Accelerator 500 Daughtercard); Via Nano Processor (e.g., L2100, L2200, U2400) line, which is capable of performing 500+MB/s of cryptographic instructions; VLSI Technology's 33 MHz 6868; and/or the like.

### Memory

Generally, any mechanization and/or embodiment allowing a processor to affect the storage and/or retrieval of information is regarded as memory 629 (or 68, 72, etc.). However, memory is a fungible technology and resource, thus, any number of memory embodiments may be employed in lieu of or in concert with one another. It is to be understood that the TSMS™ controller and/or a computer systemization may employ various forms of memory 629. For example, a computer systemization may be configured wherein the functionality of on-chip CPU memory (e.g., registers), RAM, ROM, and any other storage devices are provided by a paper punch tape or paper punch card mechanism; of course such an embodiment would result in an extremely slow rate of operation. In a typical configuration, memory 629 will include ROM 606, RAM 605, and a storage device 614. A storage device 614 may be any conventional computer system storage. Storage devices may include a drum; a (fixed and/or removable) magnetic disk drive; a magneto-optical drive; an optical drive (i.e., Blueray, CD ROM/RAM/Recordable (R)/ReWritable (RW), DVD R/RW, HD DVD R/RW etc.); an array of devices (e.g., Redundant Array of Independent Disks (RAID)); solid state memory devices (USB memory, solid state drives (SSD), etc.); other processor-readable storage mediums; and/or other devices of the like. Thus, a computer systemization generally requires and makes use of memory.

### Component Collection

The memory 629 may contain a collection of program and/or database components and/or data such as, but not limited to: operating system component(s) 615 (operating system); information server component(s) 616 (information server); user interface component(s) 617 (user interface); Web browser component(s) 618 (Web browser); database(s) 619; mail server component(s) 621; mail client component(s) 622; cryptographic server component(s) 620 (cryptographic server) and/or the like (i.e., collectively a component collection). These components may be stored and accessed from the storage devices and/or from storage devices accessible through an interface bus. Although non-conventional program components such as those in the component collection, typically, are stored in a local storage device 614, they may also be loaded and/or stored in memory such as: peripheral devices, RAM, remote storage facilities through a communications network, ROM, various forms of memory, and/or the like.

### Operating System

The operating system component 615 is an executable program component facilitating the operation of the TSMS™ controller. Typically, the operating system facilitates access of I/O, network interfaces, peripheral devices, storage devices, and/or the like. The operating system may be a highly fault tolerant, scalable, and secure system such as: Apple Macintosh OS X (Server); AT&T Plan 9; Be OS; Unix and Unix-like system distributions (such as AT&T's UNIX; Berkley Software Distribution (BSD) variations such as FreeBSD, NetBSD, OpenBSD, and/or the like; Linux distributions such as Red Hat, Ubuntu, and/or the like); and/or the like operating systems. However, more limited and/or less secure operating systems also may be employed such as Apple Macintosh OS, IBM OS/2, Microsoft DOS, Microsoft Windows 2000/2003/3.1/95/98/CE/Millenium/NT/Vista/XP (Server), Palm OS, and/or the like. An operating system may communicate to and/or with other components in a component collection, including itself, and/or the like. Most frequently, the operating system communicates with other program components, user interfaces, and/or the like. For example, the operating system may contain, communicate, generate, obtain, and/or provide program component, system, user, and/or data communications, requests, and/or responses. The operating system, once executed by the CPU, may enable the interaction with communications networks, data, I/O, peripheral devices, program components, memory, user input devices, and/or the like. The operating system may provide communications protocols that allow the TSMS™ controller to communicate with other entities through a communications network 613. Various communication protocols may be used by the TSMS™ controller as a subcarrier transport mechanism for interaction, such as, but not limited to: multicast, TCP/IP, UDP, unicast, and/or the like.

### Information Server

An information server component 616 is a stored program component that is executed by a CPU. The information server may be a conventional Internet information server such as, but not limited to Apache Software Foundation's Apache, Microsoft's Internet Information Server, and/or the like. The information server may allow for the execution of program components through facilities such as Active Server Page (ASP), ActiveX, (ANSI) (Objective-) C (++), C# and/or .NET, Common Gateway Interface (CGI) scripts, dynamic (D) hypertext markup language (HTML), FLASH, Java, JavaScript, Practical Extraction Report Language (PERL), Hypertext Pre-Processor (PHP), pipes, Python, wireless application protocol (WAP), WebObjects, and/or the like. The information server may support secure communications protocols such as, but not limited to, File Transfer Protocol (FTP); HyperText Transfer Protocol (HTIP); Secure Hypertext Transfer Protocol (HTTPS), Secure Socket Layer (SSL), messaging protocols (e.g., America Online (AOL) Instant Messenger (AIM), Application Exchange (APEX), ICQ, Internet Relay Chat (IRC), Microsoft Network (MSN) Messenger Service, Presence and Instant Messaging Protocol (PRIM), Internet Engineering Task Force's (IETF's) Session Initiation Protocol (SIP), SIP for Instant Messaging and Presence Leveraging Extensions (SIMPLE), open XML-based Extensible Messaging and Presence Protocol (XMPP) (i.e., Jabber or Open Mobile Alliance's (OMA's) Instant Messaging and Presence Service (IMPS)), Yahoo! Instant Messenger Service, and/or the like. The information server provides results in the form of Web pages to Web browsers, and allows for the manipulated generation of the Web pages through interaction with other program components. After a Domain Name System (DNS) resolution portion of an HTTP request is resolved to a particular information server, the information server resolves requests for information at specified locations on the TSMS™ controller based on the remainder of the HTTP request. For example, a request such as http://123.124.125.126/myInformation.html might have the IP portion of the request “123.124.125.126” resolved by a DNS server to an information server at that IP address; that information server might in turn further parse the http request for the “/myInformation.html” portion of the request and resolve it to a location in memory containing the information “myInformation.html.” Additionally, other information serving protocols may be employed across various ports, e.g., FTP communications across port 21, and/or the like. An information server may communicate to and/or with other components in a component collection, including itself, and/or facilities of the like. Most frequently, the information server communicates with the TSMS™ database 619, operating systems, other program components, user interfaces, Web browsers, and/or the like.

Access to the TSMS™ database may be achieved through a number of database bridge mechanisms such as through scripting languages as enumerated below (e.g., CGI) and through inter-application communication channels as enumerated below (e.g., CORBA, WebObjects, etc.). Any data requests through a Web browser are parsed through the bridge mechanism into appropriate grammars as required by the TSMS™ In one embodiment, the information server would provide a Web form accessible by a Web browser. Entries made into supplied fields in the Web form are tagged as having been entered into the particular fields, and parsed as such. The entered terms are then passed along with the field tags, which act to instruct the parser to generate queries directed to appropriate tables and/or fields. In one embodiment, the parser may generate queries in standard SQL by instantiating a search string with the proper join/select commands based on the tagged text entries, wherein the resulting command is provided over the bridge mechanism to the TSMS™ as a query. Upon generating query results from the query, the results are passed over the bridge mechanism, and may be parsed for formatting and generation of a new results Web page by the bridge mechanism. Such a new results Web page is then provided to the information server, which may supply it to the requesting Web browser.

Also, an information server may contain, communicate, generate, obtain, and/or provide program component, system, user, and/or data communications, requests, and/or responses.

### User Interface

Computer interfaces in some respects are similar to automobile operation interfaces. Automobile operation interface elements such as steering wheels, gearshifts, and speedometers facilitate the access, operation, and display of automobile resources, and status. Computer interaction interface elements such as check boxes, cursors, menus, scrollers, and windows (collectively and commonly referred to as widgets) similarly facilitate the access, capabilities, operation, and display of data and computer hardware and operating system resources, and status. Operation interfaces are commonly called user interfaces. Graphical user interfaces (GUIs) such as the Apple Macintosh Operating System's Aqua, IBM's OS/2, Microsoft's Windows 2000/2003/3.1/95/98/CE/Millenium/NT/XP/Vista/7 (i.e., Aero), Unix's X-Windows (e.g., which may include additional Unix graphic interface libraries and layers such as K Desktop Environment (KDE), mythTV and GNU Network Object Model Environment (GNOME)), web interface libraries (e.g., ActiveX, AJAX, (D)HTML, FLASH, Java, JavaScript, etc. interface libraries such as, but not limited to, Dojo, jQuery(UI), MooTools, Prototype, script.aculo.us, SWFObject, Yahoo! User Interface, any of which may be used and) provide a baseline and means of accessing and displaying information graphically to users.

A user interface component 617 is a stored program component that is executed by a CPU. The user interface may be a conventional graphic user interface as provided by, with, and/or atop operating systems and/or operating environments such as already discussed. The user interface may allow for the display, execution, interaction, manipulation, and/or operation of program components and/or system facilities through textual and/or graphical facilities. The user interface provides a facility through which users may affect, interact, and/or operate a computer system. A user interface may communicate to and/or with other components in a component collection, including itself, and/or facilities of the like. Most frequently, the user interface communicates with operating systems, other program components, and/or the like. The user interface may contain, communicate, generate, obtain, and/or provide program component, system, user, and/or data communications, requests, and/or responses.

### Web Browser

A Web browser component 618 is a stored program component that is executed by a CPU. The Web browser may be a conventional hypertext viewing application such as Microsoft Internet Explorer or Netscape Navigator. Secure Web browsing may be supplied with 128 bit (or greater) encryption by way of HTTPS, SSL, and/or the like. Web browsers allowing for the execution of program components through facilities such as ActiveX, AJAX, (D)HTML, FLASH, Java, JavaScript, web browser plug-in APIs (e.g., FireFox, Safari Plug-in, and/or the like APIs), and/or the like. Web browsers and like information access tools may be integrated into PDAs, cellular telephones, and/or other mobile devices. A Web browser may communicate to and/or with other components in a component collection, including itself, and/or facilities of the like. Most frequently, the Web browser communicates with information servers, operating systems, integrated program components (e.g., plug-ins), and/or the like; e.g., it may contain, communicate, generate, obtain, and/or provide program component, system, user, and/or data communications, requests, and/or responses. Of course, in place of a Web browser and information server, a combined application may be developed to perform similar functions of both. The combined application would similarly affect the obtaining and the provision of information to users, user agents, and/or the like from the TSMS™ enabled nodes. The combined application may be nugatory on systems employing standard Web browsers.

### Mail Server

A mail server component 621 is a stored program component that is executed by a CPU 603. The mail server may be a conventional Internet mail server such as, but not limited to sendmail, Microsoft Exchange, and/or the like. The mail server may allow for the execution of program components through facilities such as ASP, ActiveX, (ANSI) (Objective-) C (++), C# and/or .NET, CGI scripts, Java, JavaScript, PERL, PHP, pipes, Python, WebObjects, and/or the like. The mail server may support communications protocols such as, but not limited to: Internet message access protocol (IMAP), Messaging Application Programming Interface (MAPI)/Microsoft Exchange, post office protocol (POP3), simple mail transfer protocol (SMTP), and/or the like. The mail server can route, forward, and process incoming and outgoing mail messages that have been sent, relayed and/or otherwise traversing through and/or to the TSMS™.

Access to the TSMS™ mail may be achieved through a number of APIs offered by the individual Web server components and/or the operating system.

Also, a mail server may contain, communicate, generate, obtain, and/or provide program component, system, user, and/or data communications, requests, information, and/or responses.

### Mail Client

A mail client component 622 is a stored program component that is executed by a CPU 603. The mail client may be a conventional mail viewing application such as Apple Mail, Microsoft Entourage, Microsoft Outlook, Microsoft Outlook Express, Mozilla, Thunderbird, and/or the like. Mail clients may support a number of transfer protocols, such as: IMAP, Microsoft Exchange, POP3, SMTP, and/or the like. A mail client may communicate to and/or with other components in a component collection, including itself, and/or facilities of the like. Most frequently, the mail client communicates with mail servers, operating systems, other mail clients, and/or the like; e.g., it may contain, communicate, generate, obtain, and/or provide program component, system, user, and/or data communications, requests, information, and/or responses. Generally, the mail client provides a facility to compose and transmit electronic mail messages.

### Cryptographic Server

A cryptographic server component 620 is a stored program component that is executed by a CPU 603, cryptographic processor 626, cryptographic processor interface 627, cryptographic processor device 628, and/or the like. Cryptographic processor interfaces will allow for expedition of encryption and/or decryption requests by the cryptographic component; however, the cryptographic component, alternatively, may run on a conventional CPU. The cryptographic component allows for the encryption and/or decryption of provided data. The cryptographic component allows for both symmetric and asymmetric (e.g., Pretty Good Protection (PGP)) encryption and/or decryption. The cryptographic component may employ cryptographic techniques such as, but not limited to: digital certificates (e.g., X.509 authentication framework), digital signatures, dual signatures, enveloping, password access protection, public key management, and/or the like. The cryptographic component will facilitate numerous (encryption and/or decryption) security protocols such as, but not limited to: checksum, Data Encryption Standard (DES), Elliptical Curve Encryption (ECC), International Data Encryption Algorithm (IDEA), Message Digest 5 (MDS, which is a one way hash function), passwords, Rivest Cipher (RCS), Rijndael, RSA (which is an Internet encryption and authentication system that uses an algorithm developed in 1977 by Ron Rivest, Adi Shamir, and Leonard Adleman), Secure Hash Algorithm (SHA), Secure Socket Layer (SSL), Secure Hypertext Transfer Protocol (HTTPS), and/or the like. Employing such encryption security protocols, the TSMS™ may encrypt all incoming and/or outgoing communications and may serve as node within a virtual private network (VPN) with a wider communications network. The cryptographic component facilitates the process of “security authorization” whereby access to a resource is inhibited by a security protocol wherein the cryptographic component effects authorized access to the secured resource. In addition, the cryptographic component may provide unique identifiers of content, e.g., employing and MDS hash to obtain a unique signature for an digital audio file. A cryptographic component may communicate to and/or with other components in a component collection, including itself, and/or facilities of the like. The cryptographic component supports encryption schemes allowing for the secure transmission of information across a communications network to enable the TSMS™ component to engage in secure transactions if so desired. The cryptographic component facilitates the secure accessing of resources on the TSMS™ and facilitates the access of secured resources on remote systems; i.e., it may act as a client and/or server of secured resources. Most frequently, the cryptographic component communicates with information servers, operating systems, other program components, and/or the like. The cryptographic component may contain, communicate, generate, obtain, and/or provide program component, system, user, and/or data communications, requests, and/or responses.

### The TSMS™ Database

The TSMS™ database component 619 may be embodied in a database and its stored data. The database is a stored program component, which is executed by the CPU; the stored program component portion configuring the CPU to process the stored data. The database may be a conventional, fault tolerant, relational, scalable, secure database such as Oracle or Sybase. Relational databases are an extension of a flat file. Relational databases consist of a series of related tables. The tables are interconnected via a key field. Use of the key field allows the combination of the tables by indexing against the key field; i.e., the key fields act as dimensional pivot points for combining information from various tables. Relationships generally identify links maintained between tables by matching primary keys. Primary keys represent fields that uniquely identify the rows of a table in a relational database. More precisely, they uniquely identify rows of a table on the “one” side of a one-to-many relationship.

Alternatively, the TSMS™ database may be implemented using various standard data-structures, such as an array, hash, (linked) list, struct, structured text file (e.g., XML), table, and/or the like. Such data-structures may be stored in memory and/or in (structured) files. In another alternative, an object-oriented database may be used, such as Frontier, ObjectStore, Poet, Zope, and/or the like. Object databases can include a number of object collections that are grouped and/or linked together by common attributes; they may be related to other object collections by some common attributes. Object-oriented databases perform similarly to relational databases with the exception that objects are not just pieces of data but may have other types of functionality encapsulated within a given object. If the TSMS™ database is implemented as a data-structure, the use of the TSMS™ database 619 may be integrated into another component such as the TSMS™ component 635. Also, the database may be implemented as a mix of data structures, objects, and relational structures. Databases may be consolidated and/or distributed in countless variations through standard data processing techniques. Portions of databases, e.g., tables, may be exported and/or imported and thus decentralized and/or integrated.

In one embodiment, the database component 619 includes several tables 619a-j. A Users (e.g., operators and physicians) table 619a may include fields such as, but not limited to: user_id, ssn, dob, first_name, last_name, age, state, address_firstline, address_secondline, zipcode, devices_list, contact_info, contact_type, alt_contact_info, alt_contact_type, and/or the like to refer to any type of enterable data or selections discussed herein. The Users table may support and/or track multiple entity accounts. A Clients table 619b may include fields such as, but not limited to: user_id, client_id, client_ip, client_type, client_model, operating_system, os_version, app_installed_flag, and/or the like. An Apps table 619c may include fields such as, but not limited to: app_ID, app_name, app_type, OS_compatibilities_list, version, timestamp, developer_ID, and/or the like. A Patients table for patients associated with an entity administering the magnetic resonance system 619d may include fields such as, but not limited to: patient_id, patient_name, patient_address, ip_address, mac_address, auth_key, port_num, security_settings_list, and/or the like. An MR Studies table 619e may include fields such as, but not limited to: study_id, study_name, security_settings_list, study_parameters, rf_sequences, gradient_sequences, coil_selection, imaging_mode, and/or the like. An RF sequences table 619f including a plurality of different rf pulse sequences may include fields such as, but not limited to: sequence_type, sequence_id, tip_angle, coil_selection, power_level, and/or the like. A gradient sequences table 619g may include fields relating to different gradient field sequences such as, but not limited to: sequence_id, Gx, Gy, Gz, Gxy, Gxz, Gyz, Gxyz, field_strength, time_duration, and/or the like. A raw MR data table 619h may include fields such as, but not limited to: study_id, time_stamp, file_size, patient_id, rf_sequence, body_part_imaged, slice_id, and/or the like. A Images table 619i may include fields such as, but not limited to: image_id, study_id, file_size, patient_id, time_stamp, settings, and/or the like. A Payment Legers table 619j may include fields such as, but not limited to: request_id, timestamp, payment_amount, batch_id, transaction_id, clear_flag, deposit_account, transaction_summary, patient_name, patient_account, and/or the like.

In one embodiment, user programs may contain various user interface primitives, which may serve to update the TSMS™ platform. Also, various accounts may require custom database tables depending upon the environments and the types of clients the TSMS™ system may need to serve. It should be noted that any unique fields may be designated as a key field throughout. In an alternative embodiment, these tables have been decentralized into their own databases and their respective database controllers (i.e., individual database controllers for each of the above tables). Employing standard data processing techniques, one may further distribute the databases over several computer systemizations and/or storage devices. Similarly, configurations of the decentralized database controllers may be varied by consolidating and/or distributing the various database components 619a-j. The TSMS™ system may be configured to keep track of various settings, inputs, and parameters via database controllers.

The TSMS™ database may communicate to and/or with other components in a component collection, including itself, and/or facilities of the like. Most frequently, the TSMS™ database communicates with the TSMS™ component, other program components, and/or the like. The database may contain, retain, and provide information regarding other nodes and data.

### The TSMS™ Components

The TSMS™ component 635 is a stored program component that is executed by a CPU. In one embodiment, the TSMS™ component incorporates any and/or all combinations of the aspects of the TSMS™ systems discussed in the previous figures. As such, the TSMS™ component affects accessing, obtaining and the provision of information, services, transactions, and/or the like across various communications networks.

The TSMS™ component may transform raw data collected by the magnetic resonance system into an image, such as a real time image showing the position of one or more medical device(s).

The TSMS™ component enabling access of information between nodes may be developed by employing standard development tools and languages such as, but not limited to: Apache components, Assembly, ActiveX, binary executables, (ANSI) (Objective-) C (++), C# and/or .NET, database adapters, CGI scripts, Java, JavaScript, mapping tools, procedural and object oriented development tools, PERL, PHP, Python, shell scripts, SQL commands, web application server extensions, web development environments and libraries (e.g., Microsoft's ActiveX; Adobe AIR, FLEX & FLASH; AJAX; (D)HTML; Dojo, Java; JavaScript; jQuery(UI); MooTools; Prototype; script.aculo.us; Simple Object Access Protocol (SOAP); SWFObject; Yahoo! User Interface; and/or the like), WebObjects, and/or the like. In one embodiment, the TSMS™ server employs a cryptographic server to encrypt and decrypt communications. The TSMS™ component may communicate to and/or with other components in a component collection, including itself, and/or facilities of the like. Most frequently, the TSMS™ component communicates with the TSMS™ database, operating systems, other program components, and/or the like. The TSMS™ may contain, communicate, generate, obtain, and/or provide program component, system, user, and/or data communications, requests, and/or responses.

### Distributed TSMS™ Embodiments

The structure and/or operation of any of the TSMS™ node controller components may be combined, consolidated, and/or distributed in any number of ways to facilitate development and/or deployment. Similarly, the component collection may be combined in any number of ways to facilitate deployment and/or development. To accomplish this, one may integrate the components into a common code base or in a facility that can dynamically load the components on demand in an integrated fashion.

The component collection may be consolidated and/or distributed in countless variations through standard data processing and/or development techniques. Multiple instances of any one of the program components in the program component collection may be instantiated on a single node, and/or across numerous nodes to improve performance through load-balancing and/or data-processing techniques. Furthermore, single instances may also be distributed across multiple controllers and/or storage devices; e.g., databases. All program component instances and controllers working in concert may do so through standard data processing communication techniques.

The configuration of the TSMS™ controller will depend on the context of system deployment. Factors such as, but not limited to, the budget, capacity, location, and/or use of the underlying hardware resources may affect deployment requirements and configuration. Regardless of if the configuration results in more consolidated and/or integrated program components, results in a more distributed series of program components, and/or results in some combination between a consolidated and distributed configuration, data may be communicated, obtained, and/or provided. Instances of components consolidated into a common code base from the program component collection may communicate, obtain, and/or provide data. This may be accomplished through intra-application data processing communication techniques such as, but not limited to: data referencing (e.g., pointers), internal messaging, object instance variable communication, shared memory space, variable passing, and/or the like.

If component collection components are discrete, separate, and/or external to one another, then communicating, obtaining, and/or providing data with and/or to other component components may be accomplished through inter-application data processing communication techniques such as, but not limited to: Application Program Interfaces (API) information passage; (distributed) Component Object Model ((D)COM), (Distributed) Object Linking and Embedding ((D)OLE), and/or the like), Common Object Request Broker Architecture (CORBA), Jini local and remote application program interfaces, JavaScript Object Notation (JSON), Remote Method Invocation (RMI), SOAP, process pipes, shared files, and/or the like. Messages sent between discrete component components for inter-application communication or within memory spaces of a singular component for intra-application communication may be facilitated through the creation and parsing of a grammar. A grammar may be developed by using development tools such as lex, yacc, XML, and/or the like, which allow for grammar generation and parsing capabilities, which in turn may form the basis of communication messages within and between components.

For example, a grammar may be arranged to recognize the tokens of an HTTP post command, e.g.:
w3c-post http:// . . . Value1
where Value1 is discerned as being a parameter because “http://” is part of the grammar syntax, and what follows is considered part of the post value. Similarly, with such a grammar, a variable “Value1” may be inserted into an “http://” post command and then sent. The grammar syntax itself may be presented as structured data that is interpreted and/or otherwise used to generate the parsing mechanism (e.g., a syntax description text file as processed by lex, yacc, etc.). Also, once the parsing mechanism is generated and/or instantiated, it itself may process and/or parse structured data such as, but not limited to: character (e.g., tab) delineated text, HTML, structured text streams, XML, and/or the like structured data. In another embodiment, inter-application data processing protocols themselves may have integrated and/or readily available parsers (e.g., JSON, SOAP, and/or like parsers) that may be employed to parse (e.g., communications) data. Further, the parsing grammar may be used beyond message parsing, but may also be used to parse: databases, data collections, data stores, structured data, and/or the like. Again, the desired configuration will depend upon the context, environment, and requirements of system deployment.

For example, in some implementations, the TSMS™ controller may be executing a PHP script implementing a Secure Sockets Layer (“SSL”) socket server via the information server, which listens to incoming communications on a server port to which a client may send data, e.g., data encoded in JSON format. Upon identifying an incoming communication, the PHP script may read the incoming message from the client device, parse the received JSON-encoded text data to extract information from the JSON-encoded text data into PHP script variables, and store the data (e.g., client identifying information, etc.) and/or extracted information in a relational database accessible using the Structured Query Language (“SQL”). An exemplary listing, written substantially in the form of PHP/SQL commands, to accept JSON-encoded input data from a client device via a SSL connection, parse the data to extract variables, and store the data to a database, is provided below:

```
<?PHP
header(′Content-Type: text/plain′);
// set ip address and port to listen to for incoming data
$address = ‘192.168.0.100’;
$port = 255;
// create a server-side SSL socket, listen for/accept incoming
communication
$sock = socket_create(AF_INET, SOCK_STREAM, 0);
socket_bind($sock, $address, $port) or die(‘Could not bind to address');
socket_listen($sock);
$client = socket_accept($sock);
// read input data from client device in 1024 byte blocks until end of
message
do {
  $input = “”;
  $input = socket_read($client, 1024);
  $data .= $input;
} while($input != “”);
// parse data to extract variables
$obj = json_decode($data, true);
// store input data in a database
mysql_connect(″201.408.185.132″,$DBserver,$password); // access
database
server
mysql_select(″CLIENT_DB.SQL″); // select database to append
mysql_query(“INSERT INTO UserTable (transmission)
VALUES ($data)”); // add data to UserTable table in a CLIENT database
mysql_close(″CLIENT_DB.SQL″); // close connection to database
?>
```

Also, the following resources may be used to provide example embodiments regarding SOAP parser implementation:

```
http://www.xav.com/perl/site/lib/SOAP/Parser.html
http://publib.boulder.ibm.com/infocenter/tivihelp/v2r1/index.jsp?topic=/com.i
bm.IBMDI.doc/referenceguide295.htm
  and other parser implementations:
http://publib.boulder.ibm.com/infocenter/tivihelp/v2r1/index.jsp?topic=/com.i
bm.IBMDI.doc/referenceguide259.htm
  all of which are hereby expressly incorporated by reference.
```

In order to address various issues and advance the art, the entirety of this application for TSMS™ APPARATUSES, METHODS AND SYSTEMS (including the Cover Page, Title, Headings, Field, Background, Summary, Brief Description of the Drawings, Detailed Description, Claims, Abstract, Figures, Appendices and/or otherwise) shows by way of illustration various embodiments in which the disclosed embodiments may be practiced. The advantages and features of the application are of a representative sample of embodiments only, and are not exhaustive and/or exclusive. They are presented only to assist in understanding and teach the claimed principles. It should be understood that they are not representative of all disclosed embodiments. As such, certain aspects of the disclosure have not been discussed herein. That alternate embodiments may not have been presented for a specific portion of the disclosure or that further undescribed alternate embodiments may be available for a portion is not to be considered a disclaimer of those alternate embodiments. It will be appreciated that many of those undescribed embodiments incorporate the same principles of the disclosure and others are equivalent. Thus, it is to be understood that other embodiments may be utilized and functional, logical, organizational, structural and/or topological modifications may be made without departing from the scope and/or spirit of the disclosure. As such, all examples and/or embodiments are deemed to be non-limiting throughout this disclosure. Also, no inference should be drawn regarding those embodiments discussed herein relative to those not discussed herein other than it is as such for purposes of reducing space and repetition. For instance, it is to be understood that the logical and/or topological structure of any combination of any program components (a component collection), other components and/or any present feature sets as described in the figures and/or throughout are not limited to a fixed operating order and/or arrangement, but rather, any disclosed order is exemplary and all equivalents, regardless of order, are contemplated by the disclosure. Furthermore, it is to be understood that such features are not limited to serial execution, but rather, any number of threads, processes, services, servers, and/or the like that may execute asynchronously, concurrently, in parallel, simultaneously, synchronously, and/or the like are contemplated by the disclosure. As such, some of these features may be mutually contradictory, in that they cannot be simultaneously present in a single embodiment. Similarly, some features are applicable to one aspect of the disclosure, and inapplicable to others. In addition, the disclosure includes other embodiments not presently claimed. Applicant reserves all rights in those presently unclaimed embodiments including the right to claim such embodiments, file additional applications, continuations, continuations in part, divisions, and/or the like thereof. As such, it should be understood that advantages, embodiments, examples, functional, features, logical, organizational, structural, topological, and/or other aspects of the disclosure are not to be considered limitations on the disclosure as defined by the claims or limitations on equivalents to the claims. It is to be understood that, depending on the particular needs and/or characteristics of a TSMS™ individual and/or enterprise user, database configuration and/or relational model, data type, data transmission and/or network framework, syntax structure, and/or the like, various embodiments of the TSMS™ may be implemented that enable a great deal of flexibility and customization.

All statements herein reciting principles, aspects, and embodiments of the disclosure, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

Descriptions herein of circuitry and method steps and computer programs represent conceptual embodiments of illustrative circuitry and software embodying the principles of the disclosed embodiments. Thus the functions of the various elements shown and described herein may be provided through the use of dedicated hardware as well as hardware capable of executing software in association with appropriate software as set forth herein.

In the disclosure hereof any element expressed as a means for performing a specified function is intended to encompass any way of performing that function including, for example, a) a combination of circuit elements and associated hardware which perform that function or b) software in any form, including, therefore, firmware, microcode or the like as set forth herein, combined with appropriate circuitry for executing that software to perform the function. Applicants thus regard any means which can provide those functionalities as equivalent to those shown herein.

Similarly, it will be appreciated that the system and process flows described herein represent various processes which may be substantially represented in computer-readable media and so executed by a computer or processor, whether or not such computer or processor is explicitly shown. Moreover, the various processes can be understood as representing not only processing and/or other functions but, alternatively, as blocks of program code that carry out such processing or functions.

### REFERENCES

Each and every one of the below references is incorporated by reference in its entirety for all purposes.
1. Glowinski A, Kürsch J, Adam G, Bücker A, Noll T G, and Günther R W, “Device visualization for interventional MRI using local magnetic fields: basic theory and its application to catheter visualization.,” IEEE Trans. Med. Imaging, vol. 17, no. 5, pp. 786-93, 10 1998.
2. Rube M A, Holbrook A B, Cox B F, Houston J G, and Melzer A, “Wireless M R tracking of interventional devices using phase-field dithering and projection reconstruction.,” Magn. Reson. Imaging, vol. 32, no. 6, pp. 693-701, 7 2014.
3. Thorne B R H, V Lillaney P, Losey A D, and Hetts S W, “Omnidirectional mri catheter resonator and related systems, methods, and devices,” WO2015164806A1, 2015.
4. Thorne B R, Lillaney P, Losey A D, Zhang X, Pang Y, and Hetts S W, “Micro Resonant Marker for Endovascular Catheter Tracking in Interventional MRI: In Vitro Imaging at 3T,” in Proc. Intl. Soc. Mag. Reson. Med, 2014, vol. 21, no. 2013, p. 2327.
5. Quick H H et al., “Interventional magnetic resonance angiography with no strings attached: Wireless active catheter visualization,” Magn. Reson. Med, vol. 53, no. 2, pp. 446-455, 2005.
6. Kuehne T, Fahrig R, and Butts K, “Pair of resonant fiducial markers for localization of endovascular catheters at all catheter orientations,” J. Magn. Reson.

Imaging, vol. 17, no. 5, pp. 620-624, 2003.
7. Celik H, Ulutiirk A, Talh T, and Atalar E, “A catheter tracking method using reverse polarization for MR-guided interventions,” Magn. Reson. Med, vol. 58, no. 6, pp. 1224-1231, 12 2007
8. Ellersiek D et al., “A monolithically fabricated flexible resonant circuit for catheter tracking in magnetic resonance imaging,” Sensors Actuators B Chem, vol. 144, no. 2, pp. 432-436, 2 2010.
9. Kaiser M et al., “Resonant marker design and fabrication techniques for device visualization during interventional magnetic resonance imaging,” Biomed. Eng./Biomed. Tech, vol. 60, no. 2, 1 2015.
10. Ellersiek D, Fassbender H, Bruners P, Pfeffer J G, Penzkofer T, Mahnken A H, Schmitz-Rode T, Mokwa W, Schnakenberg U (2010) A monolithically fabricated flexible resonant circuit for catheter tracking in magnetic resonance imaging. Sens Actuators B: Chem 144(2):432-436.
11. Serfaty J M, Yang X, Aksit P, et al. Toward MRI guided coronary catheterization: visualization of guiding catheters, guidewires, and anatomy in real time. J Magn Reson Imaging 2000; 12(4):590-4.
12. Bakker C J, Seppenwoolde J H, Bartels L W, et al. Adaptive subtraction as an aid in M R-guided placement of catheters and guidewires. J Magn Reason Imaging 2004; 20(3):470-4.
13. Peeters J M, Seppenwoolde J H, Bartels L W, et al. Development and testing of passive tracking markers for different field strengths and tracking speeds. Phys Med Biol 2006; 51(6): N127-37.
14. Glowinski A, Adam G, Bucker A, et al. Catheter visualization using locally induced, actively controlled field inhomogeneities. Magn Reson Med 1997; 38(2):253-8.
15. Roberts T P, Hassenzahl W V, Hetts S W, et al. Remote control of catheter tip deflection: an opportunity for interventional MRI. Magn Reson Med 2002; 48(6): 1091-5.
16. Settecase F, Hetts S W, Martin A J, et al. R F heating of MRI-assisted catheter steering coils for interventional MRI. Acad Radiol 2011; 18(3):277-85.

The methods, systems, computer programs and mobile devices of the present disclosure, as described above and shown in the drawings, among other things, provide for improved magnetic resonance methods, systems and machine-readable programs for carrying out the same. It will be apparent to those skilled in the art that various modifications and variations can be made in the devices, methods, software programs and mobile devices of the present disclosure without departing from the spirit or scope of the disclosure. Thus, it is intended that the present disclosure include modifications and variations that are within the scope of the subject disclosure and equivalents.

What is claimed is:

1. A method for performing magnetic resonance imaging comprising:
a) providing a magnetic resonance device including (i) a main magnet for providing a background magnetic field along a first direction, (ii) at least one radio-frequency coil, and (iii) at least one gradient coil that can be controlled to define a region of interest;
b) positioning a medical device with at least one MRI passive or active marker thereon within a field of view of the at least one resonant coil, the medical device being configured to emit a signal either passively or actively;
c) providing a circuit that generates pulsed DC current or AC current to act as a signal signature for the MRI passive or active marker on the medical device, the circuit being further configured to adjust signal parameters including at least one of the signal amplitude, pulse shape and signal repetition rate relative to MR scan sequence parameters to permit the at least one MRI passive or active marker to emit a signal to facilitate detection of the medical device;
d) introducing a sample or subject to be studied into the field of view;
e) introducing RF pulses into the sample or subject;
f) collecting data for a set of nuclei of interest from the sample or subject, and the passive or active marker;
g) processing the data to determine the spatial location of the medical device based on the passive or active marker; and
h) forming image data showing a relative location of the medical device with respect to surrounding anatomy.

2. The method of claim 1, wherein the same medical device is configured to be used in different magnetic field strengths and different MR sequences without sacrificing medical image quality and precision for detecting device position.

3. A method of performing magnetic resonance imaging that includes delivering pulsed DC current or AC current waveforms to create a dedicated signature in the form of magnetic field inhomogeneity at a location of a conductive markers on a medical device located within a magnetic resonance imaging device.

4. The method of claim 3, wherein pulsed DC or AC current is delivered in a manner that accounts for the TR/TE values of a MRI imaging sequence.

5. The method of claim 4, further comprising applying an algorithm to a signal trail received at a magnetic resonance device to identify the magnetic field inhomogeneities.

6. The method of claim 3, wherein the magnetic field inhomogeneities are dynamically adjustable magnetic field disruptions.

7. The method of claim 6, wherein the dynamically adjustable magnetic field disruptions are interspersed in predetermined intervals within MR signals received by the magnetic resonance device.

8. The method of claim 5, wherein, when the signals that are generated by the conductive markers are detected by the algorithm, the spatial location of these magnetic field inhomogeneities are then determined in real time.

9. The method of claim 8, wherein image reconstruction software of the magnetic resonance device is configured with instructions to colorize and/or superimpose an image of the medical device on the anatomical image after transforming received MRI signals to medical images during real time MRI.

10. The method of claim 9, wherein different marker signatures can be created and switched during real time MRI.

11. The method of claim 10, wherein a physician can turn off the passive marker signal to view the anatomical image without any magnetic field disruption.

12. A method according to claim 1, wherein a low pulsed DC current is applied to the medical device or marker in the range of 1-1000 Hz.

13. A method according to claim 1, wherein the low pulsed DC current is between 50-200 mA.

14. A method according to claim 1, wherein a low pulsed AC current is applied to the medical device or marker in the range of 1-1000 Hz.

15. A method according to claim 14 wherein the low pulsed AC current is between 50-200 mA.

16. The method of claim 1, wherein current applied to the medical device or marker is applied using a frequency outside of the imaging bandwidth but within the MRI receiver coil bandwidth to create a predetermined signal signature for the magnetic field inhomogeneities.

17. The method of claim 1, wherein pulsed DC or AC current is between about 0.5V and about 12.0V.

18. The method of claim 1, wherein software used to detect the presence of medical devices or markers utilize a machine learning (“ML”) algorithm to look for dynamically adjustable magnetic field disruptions in the imaging plane or specific frequency shift in k-space domain.

19. A system to perform the methods of claim 1.

20. A non-transitory computer readable medium storing a computer program to operate a MRI system comprising at least a main magnet for providing a background magnetic field along a first direction, at least one radio-frequency coil, and at least one gradient coil that can be controlled to define a region of interest, wherein the computer program comprises instructions to perform the method of claim 1.
